(19) United States
(12) Patent Application Publication
(10) Pub. No.: US 2006/0198909 A1
(43) Pub. Date: Sep. 7, 2006

(54) PROCESS TO LOSE WEIGHT, DRAIN UNWANTED BODY FATS AND OTHER TOXINS

(76) Inventor: Juanito B Calagui, Passaic, NJ (US)

Correspondence Address: WALTER J, TENCZA JR., 10 STATION PLACE, SUITE 3, METUCHEN, NJ, 08840, US

(21) Appl. No.: 11/306,610

(22) Filed: Jan. 4, 2006

Related U.S. Application Data
(63) Continuation-in-part of application No. 10/662,608, filed on Sep. 15, 2003, now abandoned.
(63) Continuation-in-part of application No. 10/601,684, filed on Jun. 23, 2003, now abandoned.

(51) Int. Cl. A61K 36/82

(52) U.S. Cl. 424/729

(57) ABSTRACT

A method is disclosed comprising placing about thirty-two fluid ounces of water into a receptacle, placing the receptacle with water on a burner and bringing the water to a boil, ceasing to heat the water, adding about one teaspoonful or five grams of tea beverage flavorant to the water; and brewing the combination of the water and the tea beverage flavorant for about three to five minutes. The method may further include filtering out any solid tea in the combination of water and tea beverage flavorant, and consuming the combination of the water and the tea beverage flavorant.

### CROSS REFERENCE TO RELATED APPLICATIONS

The present application is a continuation in part of and claims the priority of patent application Ser. No. 10/601,684 entitled “PROCESS TO LOSE WEIGHT, DRAIN UNWANTED BODY FATS AND OTHER TOXINS”, inventor and applicant Juanito B. Calagui, filed on Jun. 23, 2003 and the present application is also a continuation in part of and claims the priority of patent application Ser. No. 10/662,608 entitled “PROCESS TO LOSE WEIGHT, DRAIN UNWANTED BODY FATS AND OTHER TOXINS”, inventor and applicant Juanito B. Calagui, filed on Sep. 15, 2003.

### FIELD OF THE INVENTION

This invention relates to improved methods and apparatus concerning losing weight.

### BACKGROUND OF THE INVENTION

It is very common practice for kids, teenagers and adults to keep eating and eating whenever foods are available. This causes them to become overweight. Overweight individuals use diet drinks, diet pills or join physical fitness centers to lose weight and to drain unwanted body fats. However, many of these options do not result in loss of weight for many overweight people.

### SUMMARY OF THE INVENTION

The present invention in one or more embodiments provides a process which cleanses and dissolves foreign bodies within the human body.

A method is disclosed comprising the steps of placing about thirty-two fluid ounces of water into a receptacle, placing the receptacle with water on a burner and bringing the water to a boil, ceasing to heat the water, adding about one teaspoonful or five grams of tea beverage flavorant to the water; and brewing the combination of the water and the tea beverage flavorant for about three to five minutes. The method may further include filtering out any solid tea in the combination of water and tea beverage flavorant, and consuming the combination of the water and the tea beverage flavorant.

The temperature of the combination of the water and the tea beverage flavorant at the time that the water and the tea beverage flavorant are consumed may be between one hundred forty and one hundred sixty degrees Fahrenheit. The combination of the water and the tea beverage flavorant may be placed into a disposable plastic bottle.

The steps of the method, including the consumption of the mixture, may be performed, twice a day, typically once before breakfast and once before dinner. The inventor has found that such a regimen results in a good overall cleansing effect and in a loss of weight.

A mixture of water and a tea beverage flavorant together in the proportions of about thirty-two fluid ounces for about every one teaspoonful or every five grams of tea beverage flavorant can be placed into a disposable plastic bottle. The mixture can be later heated and consumed.

The present invention also provides an apparatus comprising a disposable plastic bottle, which has a mixture of water and a tea beverage flavorant in the proportions of about thirty-two fluid ounces of water for about every one teaspoonful or every five grams of tea beverage flavorant, located inside the disposable plastic bottle.

### BRIEF DESCRIPTION OF THE DRAWINGS

FIG. 1 shows a flow chart of a method in accordance with an embodiment of the present invention;

FIG. 2 shows a disposable plastic bottle which includes a mixture of water and tea beverage flavorant; and

FIG. 3 shows a flow chart of a method in accordance with another embodiment of the present invention.

### DETAILED DESCRIPTION OF THE DRAWINGS

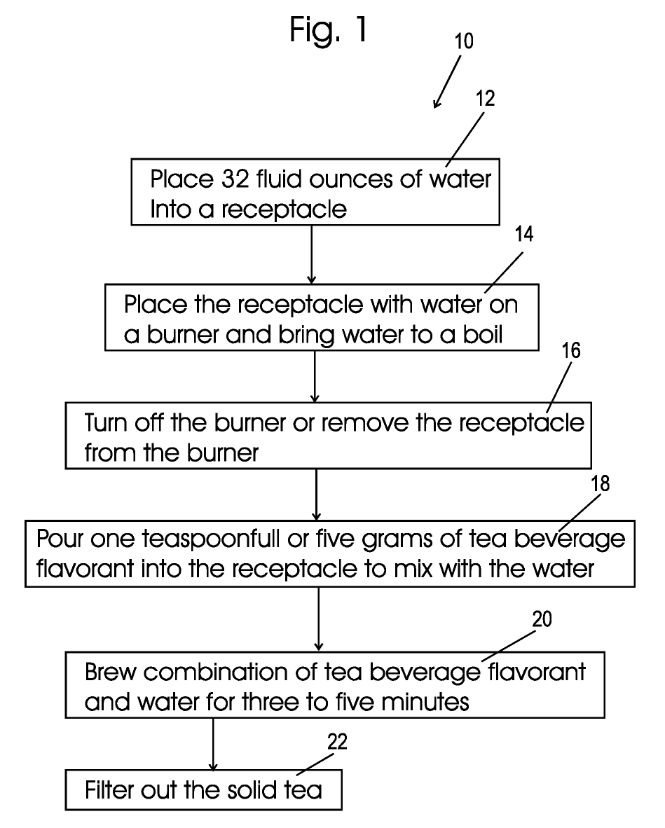

FIG. 1 shows a flow chart 10 of a method in accordance with an embodiment of the present invention. The method begins with placing thirty-two fluid ounces of water into a receptacle at step 12. The receptacle, with the water, is then placed on a burner and brought to a boil at step 14. The burner is turned off at step 16 or the receptacle with water is removed from the burner. At step 18 one teaspoonful of tea beverage flavorant or five grams is poured into the receptacle to mix with the water. At step 20 the combination of water and tea beverage flavorant is brewed for three to five minutes. At step 22 the solid tea in the combination of water and tea beverage flavorant is filtered out. The remaining combination or drink can be consumed by an individual.

The remaining drink should be at 140-160 degrees Fahrenheit when consumed by an individual for the best cleansing effect for dissolving foreign bodies and for loss of weight generally. The applicant has tested a remaining drink with the temperature between 140-160 degrees Fahrenheit versus drinks of lower temperatures and has found that the temperature range of 140-160 degrees Fahrenheit is optimum for dissolving toxins and cleansing of the body and for loss of weight.

A method in accordance with an embodiment of the present invention may include repeating the procedure shown in FIG. 1, everyday, twice a day, typically once before breakfast and once before dinner.

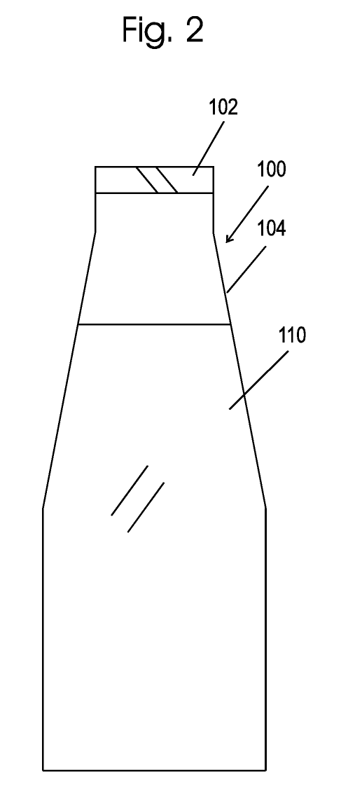

In addition, the method may additionally include placing the mixture of a ratio of 32 fluid ounces of water to one teaspoonful or five grams of tea flavorant into a disposable plastic bottle, which can be sold to a purchaser. FIG. 2 shows a disposable plastic bottle 100. The bottle 100 is transparent in this example and includes a cap 102 and a body portion 104 or receptacle for holding liquid. The body portion 104 in FIG. 2 holds a mixture of water and tea beverage flavorant 110 in the proportions of thirty-two fluid ounces of water for every one teaspoonful or every five grams of tea beverage flavorant. The cap 102 provides a seal so that the mixture 110 is held in a sealed, enclosed chamber inside the bottle 100. The mixture 110 in the disposable plastic bottle 100 can be readily heated to the appropriate temperature and consumed.

In one embodiment, draining unwanted foreign bodies may follow eight to twelve days after the process has been started. In one embodiment, draining unwanted foreign bodies may follow sixteen to twenty-one days after the process has been started.

The present invention in one or more embodiments, introduces a new process to lose weight, drain unwanted body fats and other toxins to cleanse within a living structure the human body to be healthy, pain free and cancer free.

The human body is a living structure of multiple mechanical assembly links and associated to the function of each or the other assembly. The above-mentioned linkage and association of the assembly within the human body are through the blood vessels or flowing channels and within these channels malfunctions and other irregularities are mostly caused by the buildup of fats likewise, the buildup of fats will eventually develop into the blockade of the blood vessels or flowing channels and the blockade will cause the weakest tissue within an organ to dehydrate and collapse or die and in the end decaying tissue will then turn into toxin to cause cancer. The new process is to dissolve the buildup of fats within the blood vessels or flowing channels of a human being and to dissolve pockets of fats in other portions of the body of a human being. The process in accordance with one or more embodiments of the present invention act as a cleansing agent and is effective to drain unwanted body fats and other toxins to help an individual to be healthy and to enjoy a better life.

The present invention in one or more embodiments has a beneficial effect on the heart, the respiratory system, and helps individuals to be cancer free with the use of a new process in accordance with one or more embodiments of the present invention.

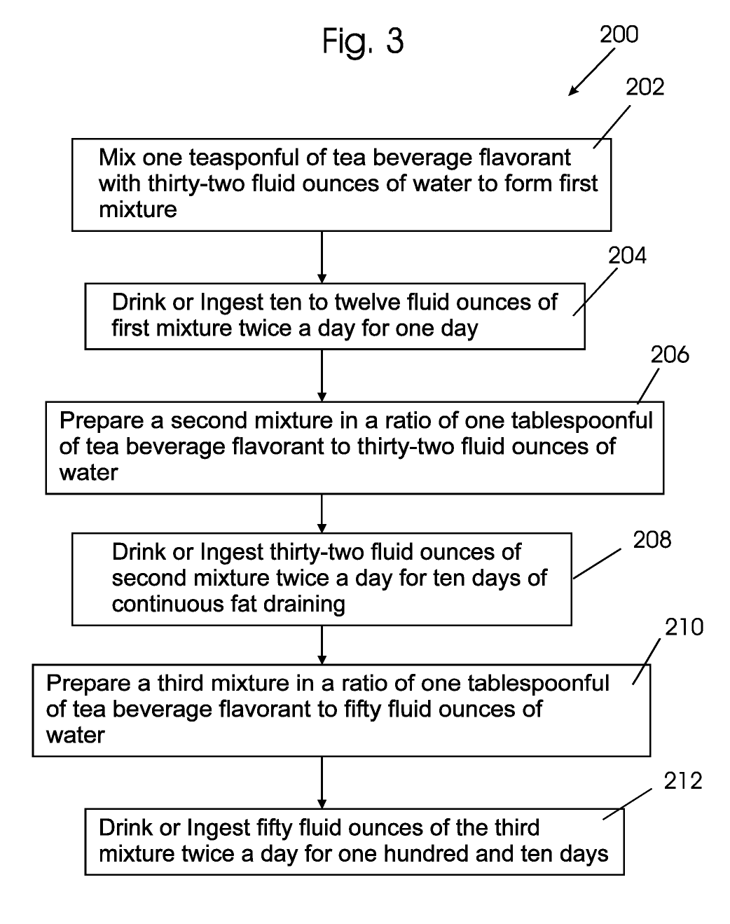

FIG. 3 shows a flow chart 200 of a method in accordance with another embodiment of the present invention. At step 202 one teaspoonful of tea beverage flavorant is mixed with about thirty-two fluid ounces of water to form a first mixture. A human being would then drink or otherwise ingest, at step 204, about ten to twelve fluid ounces of the first mixture twice a day for one day during a first or testing phase. Typically the human being would ingest or drink about ten to twelve fluid ounces of the first mixture once in the morning and once in the afternoon. The first or testing phase tests whether a human body of the human being accepts or rejects the first mixture or first tea mixture. The human being may get sick in response to the testing phase which is a sign that the human body is rejecting the first mixture.

During a phase two, at step 206 a second mixture is prepared in a ratio of one tablespoonful of tea beverage flavorant to about thirty-two fluid ounces of water. A human being would then drink or otherwise ingest, at step 208, about thirty-two fluid ounces of the second mixture twice a day until the fat starts to drain continuously for ten days during the second phase. Phase two results in the draining of fats and toxins from muscles, organs, and other portions of the human body of the human being.

At step 210, a third mixture is prepared in a ratio of one tablespoonful of tea beverage flavorant to fifty fluid ounces of water. A human being would then drink or otherwise ingest about fifty fluid ounces of the third mixture about twice a day for about one hundred and ten days during a third phase. Phase three also eliminates bad breath and drains fats from the bones of the arms and the legs of a human being.

One or more embodiments of the present invention can be used for medical and dental reasons and as preventive maintenance to help a human being to be pain free, cancer free, and healthy. It is also beneficial for people suffering from asthma. It is often difficult to detect why sicknesses, such as cancer and diabetes occur, and embodiments of the present invention are useful in ameliorating those sicknesses.

Each of the first, second, or third mixtures or teas is typically heated to a temperature of between one hundred forty and one hundred sixty degrees Fahrenheit.

The three phase procedure shown in FIG. 3 controls damage caused by diabetes and stabilizes diabetes.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

What is claimed is:

1. A method comprising the steps of:
placing about thirty-two fluid ounces of water into a receptacle;
placing the receptacle with water on a burner and bringing the water to a boil;
ceasing to heat the water;
adding about one teaspoonful of tea beverage flavorant to the water; and
brewing the combination of the water and the tea beverage flavorant for about three to five minutes.

2. The method of claim 1 further comprising
filtering out any solid tea in the combination of water and tea beverage flavorant.

3. The method of claim 1 further comprising
consuming the combination of the water and the tea beverage flavorant.

4. The method of claim 3 wherein
the temperature of the combination of the water and the tea beverage flavorant at the time that the water and the tea beverage flavorant are consumed is between one hundred forty and one hundred sixty degrees Fahrenheit.

5. The method of claim 1 further comprising
placing the combination of the water and the tea beverage flavorant into a disposable plastic bottle.

6. The method of claim 1 further comprising
performing the previous steps once before breakfast.

7. The method of claim 1 further comprising
performing the previous steps once before dinner.

8. The method of claim 1 further comprising
performing the previous steps once before breakfast and once before dinner.

9. A method comprising the steps of
mixing water and a tea beverage flavorant together in the proportions of about thirty-two fluid ounces for about every one teaspoonful of tea beverage flavorant to form a mixture;
and placing the mixture in a disposable plastic bottle.

10. A method comprising the steps of
mixing water and a tea beverage flavorant together in the proportions of about thirty-two fluid ounces for about every one teaspoonful of tea beverage flavorant to form a mixture;
and consuming the mixture.

11. An apparatus comprising
a disposable plastic bottle; and
a mixture of water and a tea beverage flavorant in the proportions of about thirty-two fluid ounces of water for about every one teaspoonful of tea beverage flavorant;
and wherein the mixture is located inside the disposable plastic bottle.

12. A method comprising the steps of:
placing about thirty-two fluid ounces of water into a receptacle;
placing the receptacle with water on a burner and bringing the water to a boil;
ceasing to heat the water;
adding about five grams of tea beverage flavorant to the water; and
brewing the combination of the water and the tea beverage flavorant for about three to five minutes.

13. The method of claim 12 further comprising filtering out any solid tea in the combination of water and tea beverage flavorant.

14. The method of claim 12 further comprising consuming the combination of the water and the tea beverage flavorant.

15. The method of claim 14 wherein
the temperature of the combination of the water and the tea beverage flavorant at the time that the water and the tea beverage flavorant are consumed is between one hundred forty and one hundred sixty degrees Fahrenheit.

16. The method of claim 12 further comprising
placing the combination of the water and the tea beverage flavorant into a disposable plastic bottle.

17. A method comprising the steps of
mixing water and a tea beverage flavorant together in the proportions of about thirty-two fluid ounces for about every five grams of tea beverage flavorant to form a mixture;
and placing the mixture in a disposable plastic bottle.

18. A method comprising the steps of
mixing water and a tea beverage flavorant together in the proportions of about thirty-two fluid ounces for about every five grams of tea beverage flavorant to form a mixture;
and consuming the mixture.

19. An apparatus comprising
a disposable plastic bottle; and
a mixture of water and a tea beverage flavorant in the proportions of about thirty-two fluid ounces of water for about every five grams of tea beverage flavorant;
and wherein the mixture is located inside the disposable plastic bottle.

20. A method comprising
during a first phase, ingesting into a human body about ten to twelve fluid ounces of a first mixture about twice a day for one day, wherein the first mixture is in a ratio of about one teaspoonful of tea beverage flavorant to thirty-two fluid ounces of water;
during a second phase, ingesting into the human body about thirty-two fluid ounces of a second mixture twice a day for about ten days, wherein the second mixture is in a ratio of about one tablespoonful of tea beverage flavorant to thirty-two fluid ounces of water; and
during a third phase, ingesting into the human body about fifty fluid ounces of a third mixture twice a day for about one hundred and ten days, wherein the third mixture is in a ratio of about one tablespoonful of tea beverage flavorant to fifty fluid ounces of water.

21. The method of claim 20 wherein
the first, second, and third mixtures are heated to between one hundred forty and one hundred sixty degrees prior to ingesting.
